(19) United States
(12) Patent Application Publication
(10) Pub. No.: US 2023/0024573 A1
(43) Pub. Date: Jan. 26, 2023

(54) TEMPORAL DISEASE STATE COMPARISON USING MULTIMODAL DATA

(71) Applicant: KONINKLIJKE PHILIPS N.V., EINDHOVEN (NL)

(72) Inventors: Kathy Mi Young LEE, WESTFORD, MA (US); Ashequl QADIR, MELROSE, MA (US); Claire Yunzhu ZHAO, BOSTON, MA (US); Minnan XU, CAMBRIDGE, MA (US); Jonathan RUBIN, CAMBRIDGE, MA (US); Nikhil GALAGALI, MOUNTAIN VIEW, MA (US)

(21) Appl. No.: 17/785,087

(22) PCT Filed: Dec. 10, 2020
(86) PCT No.: PCT/EP2020/085428

§ 371 Date: Jun. 14, 2022

Related U.S. Application Data
(60) Provisional application No. 62/949,544, filed on Dec. 18, 2019.

(51) Int. Cl. G06T 7/00; G16H 50/20

(52) U.S. Cl. CPC G06T 7/0012; G16H 50/20; G06T 2207/30004

(57) ABSTRACT

A system and method for visualizing and annotating temporal trends of an abnormal condition in patient data. A classification and visualization module detects one or more conditions in one or more images, e.g. X-ray images, and visualizes the condition on the image. A temporal disease state extraction module analyzes text, e.g. radiology reports, for indications of a change in the condition. A multimodal disease state comparison module fuses the extracted data into a compact representation of the condition changes over time.

### BACKGROUND

In critical care, abnormal conditions change at a fast rate and it is important to monitor trends in the abnormal condition very closely. To track changes in a medical condition, multiple images are taken over time and compared to prior images to identify the temporal trend, i.e., whether the condition has worsened, improved, been resolved or not changed.

In current workflows, comparing two images for a patient (e.g. chest x-rays (CXRs)) taken over a time span may require placing the images side-by side to identify temporal changes in one or more medical conditions. It is impractical for clinicians, such as an intensive care unit (ICU) physician, to observe the changes from the two images, especially when multiple diseases and comorbidities are present. When the changes are not apparent, the clinician will need to read CXR notes (written by radiologists) and find the section that describes the changes. This time-consuming process may be burdensome, particularly in ICUs where rapid interventions are required. In addition, an ICU physician's interpretation of the two images could differ from that of the radiologist. In such cases, the physician may order further test/investigation to ensure coherence of interpretation, further delaying a diagnosis and treatment.

### SUMMARY

The exemplary embodiments are directed to a method including analyzing a first image to determine whether an abnormal medical condition is present in the first image and enhancing the first image with a first heat map indicating a probability of a presence of the abnormal medical condition in an area of the first image. The method further includes analyzing a second image to determine whether the abnormal medical condition is present in the second image and enhancing the second image with a second heat map indicating a probability of a presence of the abnormal medical condition in an area of the second image. The method further includes analyzing a text-based report corresponding to the second image to extract information corresponding to the second image and changes in the abnormal medical condition between the first image and the second image. The method further includes displaying the enhanced first image, the enhanced second image and the extracted information on a single display.

The exemplary embodiments are further directed to a computer readable storage medium comprising a computer program that when executed by a processor performs the above-described method.

The exemplary embodiments are further directed to a system including a memory configured to store a plurality of imaging studies, at least a portion of the imaging studies comprising an image and a corresponding text-based image report. The system further includes a processor configured to perform operations comprising analyzing a first image of a first one of the plurality of imaging studies to determine whether an abnormal medical condition is present in the first image, enhancing the first image with a first heat map indicating a probability of a presence of the abnormal medical condition in an area of the first image, analyzing a second image of a second one of the plurality of imaging studies to determine whether the abnormal medical condition is present in the second image, enhancing the second image with a second heat map indicating a probability of a presence of the abnormal medical condition in an area of the second image, and analyzing a corresponding text-based report of the second one of the imaging studies to extract information corresponding to the second image and changes in the abnormal medical condition between the first image and the second image. The system further includes a display configured to display the enhanced first image, the enhanced second image and the extracted information.

### BRIEF DESCRIPTION OF THE DRAWINGS

FIG. 1 shows a system for visualizing and annotating temporal trends of an abnormal condition in patient data according to various exemplary embodiments of the present disclosure.

FIG. 2 shows an exemplary image and an enhanced image generated by processing the image with a classification and visualization module.

FIG. 3 shows an exemplary flow describing the functionality of a temporal disease extraction module.

FIG. 4 shows an exemplary disease state comparison showing two enhanced images representing chest x-ray images taken at different time points and including context information regarding the temporal disease state.

FIG. 5 shows an exemplary disease state comparison for a plurality of conditions.

FIG. 6 shows an exemplary temporal progression of a condition from a time prior to the condition appearing to a time when the condition has been resolved.

FIG. 7 shows a flow diagram of a method for generating a temporal disease comparison according to various exemplary embodiments of the present disclosure.

### DETAILED DESCRIPTION

The exemplary embodiments may be further understood with reference to the following description and the appended drawings, wherein like elements are referred to with the same reference numerals. The exemplary embodiments relate to systems and methods for visualizing and annotating temporal trends of an abnormal condition in patient data, the patient data including a plurality of imaging studies taken over a time span. In one embodiment, a compact representation of the temporal trends is shown in a timeline. The exemplary embodiments identify an abnormal condition in images and visualize the condition. Additionally, the exemplary embodiments extract relevant information from radiology reports associated with the images, including dates and phrases indicating changes in the condition over time, using natural language processing (NLP), machine learning (ML) or deep learning (DP) techniques. The multimodal information is combined in a temporal disease state comparison for visualizing trends in the abnormal condition.

In addition, structured information (e.g., date, condition severity, etc.), and temporal progression, may be used to train further diagnostic models such as disease progression models and temporal state change prediction models. For example, the annotated information produced for the given condition may be used to tune a disease progression model and generate predictive information about the evolution of the disease.

The exemplary embodiments will be described with reference to the images being chest (CXR) images. However, it should be understood that the use of CXR images are only exemplary and the exemplary embodiments may be applied to any type of images or image modality. Furthermore, the exemplary embodiments are also described with reference to using a convolutional neural network (CNN) based class activation mapping algorithm for visualizing an abnormal condition in the images. Once again, the use of the CNN-based method is only exemplary and any known visualization method may be employed in regard to the exemplary embodiments.

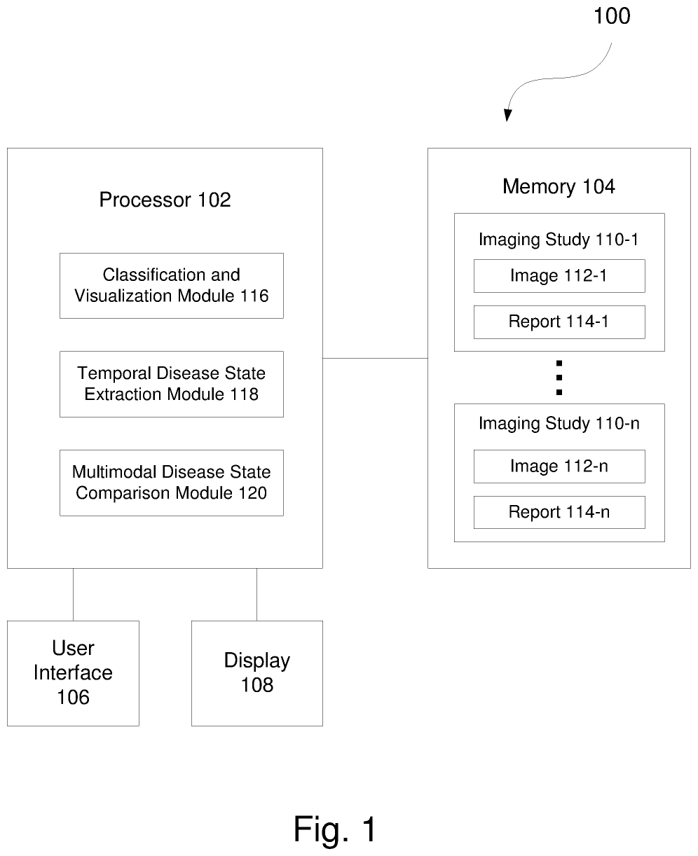

FIG. 1 shows a system 100 for visualizing and annotating temporal trends of an abnormal condition in patient data according to various exemplary embodiments of the present disclosure. The system 100 includes a processor 102 for performing various data extractions from imaging studies 110 and generating the temporal trends for the data, to be described in detail below. The system 100 further includes a memory 104 for storing patient data, specifically at least two imaging studies 110 for a patient taken over a time span. However, the patient data may have any number of imaging studies 110-1-110-n. For example, some patients may have dozens or hundreds of imaging studies 110, with all or part of those studies relating to a particular condition. The imaging studies 110 include at least one image 112 and an accompanying radiology report 114 interpreting the image(s) 112.

The system 100 includes a user interface 106 for entering a patient condition into the system 100. The system 100 may then analyze the imaging studies 110 for that condition to generate a temporal state comparison and present the comparison to a user, e.g. clinician, on a display 108. The display 108 is configured to display enhanced images (e.g., enhanced x-ray images) with accompanying textual context.

The processor 102 performs three processing functions implemented by modules, e.g., software components making up one or more programs. The processing functions each being an application (e.g., a program) executed by the processor 102 is only exemplary. The functionality associated with the processing functions may also be represented as a separate incorporated component or may be a modular component, e.g., an integrated circuit with or without firmware. For example, the integrated circuit may include input circuitry to receive signals and processing circuitry to process the signals and other information. The processing functions may also be embodied as one application or separate applications. In addition, the functionality described for the processor 102 may be split among two or more processors. The exemplary embodiments may be implemented in any of these or other configurations.

A first module implemented by the processor 102 is a classification and visualization module 116 for identifying an abnormal condition in an image 112 and visualizing the condition in an enhanced image. The classification and visualization module 116 enhances the image by providing a heat map representing a probability that a given region in the image includes the condition. The classification and visualization module 116 also tags the enhanced image with the condition and a date on which the image was acquired.

The classification and visualization module 116 first detects an abnormal medical condition, e.g., a disease, in the image 112. The classification and visualization module 116 utilizes, for example, a CNN-based class activation mapping model such as Grad-CAM, to detect the disease in the image 112. The classification and visualization module 116 may be provided with a condition or a class of conditions via the UI 106, causing the processor 102 to process the image 112 through the CNN to obtain a raw score for each pixel indicating a probability that the particular pixel belongs to the class, i.e., that the condition is present at the pixel location. Alternately, the clinician may choose not to specify a particular condition, causing the processor 102 to simultaneously detect and analyze for all conditions relevant to the anatomy shown in the image 112.

Once the image 112 has been analyzed and scored based on a probability that each of the pixels belongs to the analyzed class of conditions (or to any condition), the classification and visualization module 116 next applies the scores to the image 112 to generate a heat map. The heat map represents the scores by overlaying the image 112 with varying colors, shades of color and intensities of color. For example, a region of the image 112 having a high probability of belonging to the condition may be visualized with the color red, while a region of the image 112 having a low probability of belonging to the condition may be visualized with the color blue, with varying colors and shades of color therebetween representing regions with intermediate probabilities. A region with an especially high probability of belonging to the condition may be visualized with a variation of red color having greater intensity than a region with a moderately high probability, and a region with an especially low probability of belonging to the condition may be visualized with a variation of blue color having a greater intensity than a region with a moderately low probability.

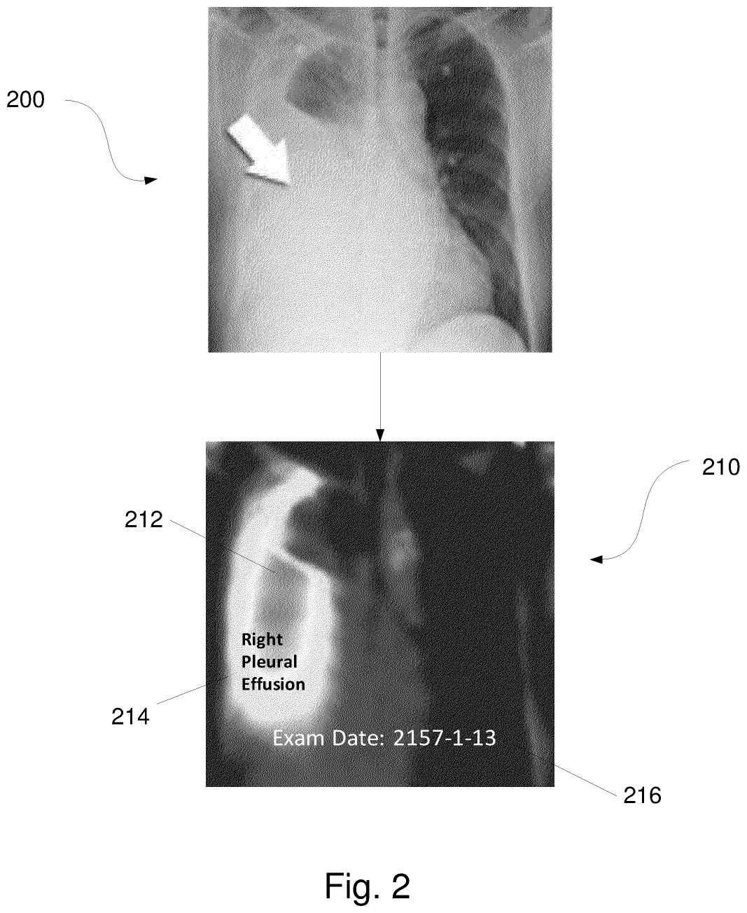

In addition to the above-described features, the classification and visualization module 116 also tags the image 112 with the name of the condition and additionally tags the image with the date of the exam. FIG. 2 shows an exemplary x-ray image 200 and an enhanced image 210 after the image 200 is processed with the classification and visualization module 116. The image 200 has been analyzed to identify regions showing a right pleural effusion. The enhanced image 210 has a heat map 212 showing the regions where the right pleural effusion may be found, with varying colors, shades and intensities as described above. The image 210 is tagged with a condition 214 (right pleural effusion) and an exam date 216. When the system 100 is analyzing for any condition, and multiple conditions are detected, the classification and visualization module 116 generates enhanced images for each image where a condition was detected.

The classification and visualization module 116 may perform the above-described image analysis for any number of images. For example, if a clinician is interested only in changes between a current scan and a most recent scan, then only those two image studies may be analyzed. However, if the clinician is interested in generating a full temporal scale of the disease, then multiple images representing different time points for the condition may be analyzed. The clinician may also select a particular time period for the temporal analysis.

When a full temporal analysis is desired, the classification and visualization module 116 may analyze those imaging studies 110 that are classified as containing images 112 relevant to the region of interest. For example, if only CXR images are desired, then the classification and visualization module 116 will first identify those imaging studies 110 containing CXR images using DICOM tagging and subsequently analyze the images 112 within.

A second module implemented by the processor 102 is a temporal disease extraction module 118 for extracting relevant information, i.e. text, from radiologist reports 114 relating to the analyzed images. The temporal disease extraction module 118 utilizes an NLP model that may be a named entity recognition (NER) model (e.g., conditional random fields (CRF) or a bidirectional Long Short-Term Memory (BI-LSTM) network with a CRF layer (BI-LSTM-CRF model)) to extract phrases describing temporal changes in the condition between a current exam and a prior exam. For example, the NLP model may identify and extract phrases describing the condition as worsened, unchanged, improved, resolved, new, etc. In addition, the temporal disease extraction module 118 extracts the condition itself, the date of the current exam, and the date of the past exam to which the current exam is being compared for use in the temporal disease state comparison.

The NLP model may receive a set of training data including radiology reports that have been manually labeled. For example, the reports may have manual labels for words or phrases describing conditions (e.g., edema, cardiomegaly, pleural effusion, pneumothorax, etc.), temporal states (e.g., new, unchanged, worsened, improved, resolved, etc.), location (right, left, upper, lower, etc.) and dates (e.g. 2168-06-30). The training data may be used to train the NER model (e.g., the BiLSTM-CNN—CRF model) to detect the aforementioned concepts from new/unseen radiology reports. Additionally, a relationship extraction (RE) model may be trained to identify which temporal state or location word is associated with which condition.

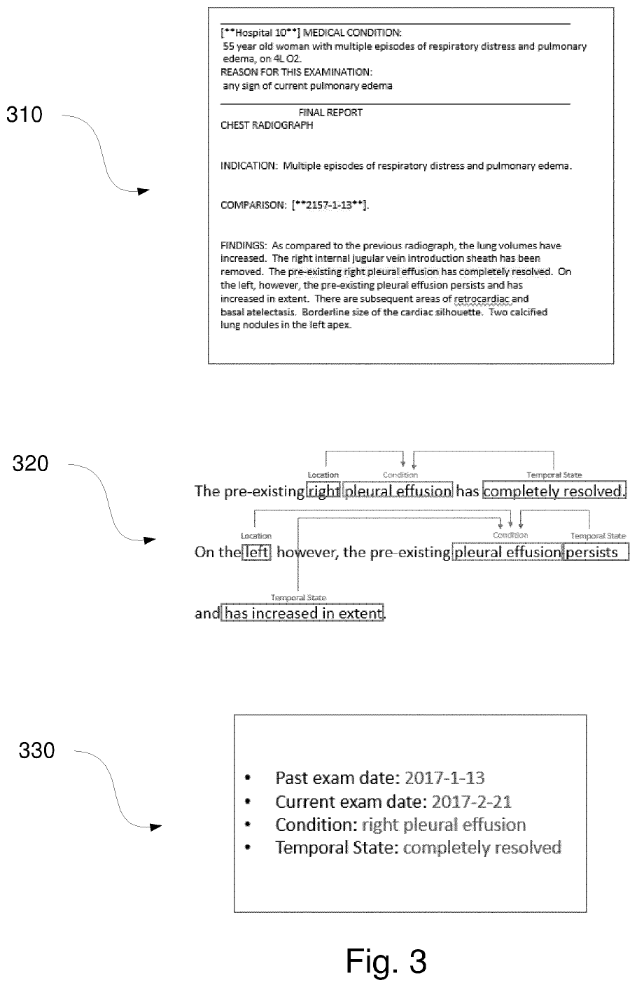

For example, FIG. 3 shows an exemplary flow describing the functionality of the temporal disease extraction module 118. An exemplary chest x-ray report 310 (typically written by a radiologist) is shown, the report 310 including the medical condition, the reason for the condition, a previous exam date, the findings, etc. The report 310 is processed with the NLP model to extract relevant concepts from the report, such as those shown in 320. The NER model can detect ‘right’ is a location word, ‘pleural effusion’ is a condition, ‘completely resolved’ is a temporal state, and the RE model can detect ‘right’ modifies ‘pleural effusion’ and the temporal state ‘completely resolved’ also modifies ‘pleural effusion’. Similarly, the NER model can detect ‘left’ is a location word, ‘pleural effusion’ is a condition, and that ‘persists’ and ‘has increased in extent’ are temporal states. Then the relation extraction model can detect the location word ‘left’ and the temporal state words ‘persists’ and ‘has increased in extent’ modify the condition ‘pleural effusion’ in the same sentence.

The extracted concepts 330 relating to the right pleural effusion are shown as i) the past exam date, ii) the current exam date, iii) the condition and iv) the temporal state, for use in a temporal disease state comparison.

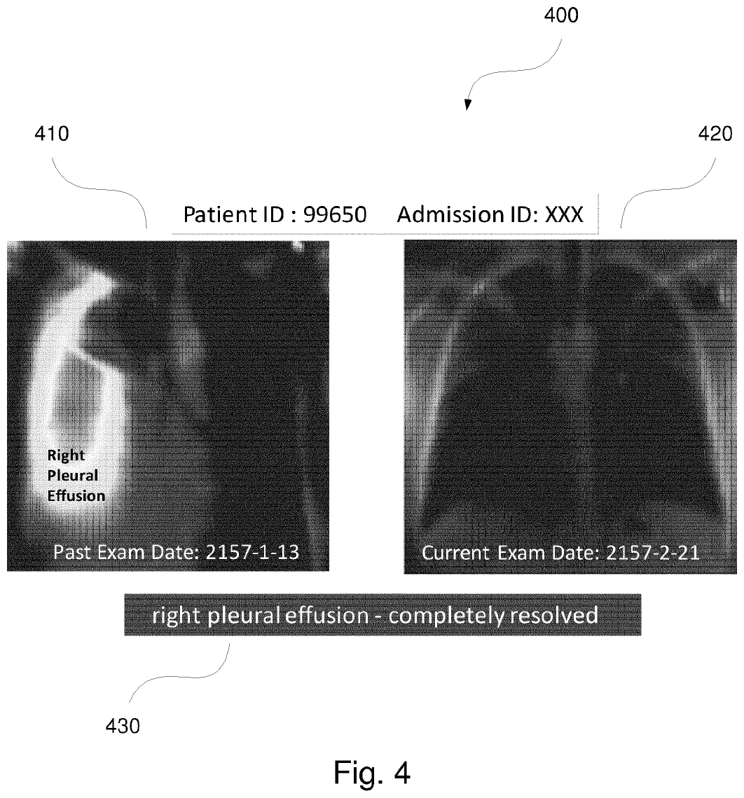

A third module implemented by the processor 102 is a multimodal disease state comparison module 120. The comparison module 120 fuses the data extracted from the classification and visualization module 116 and the temporal disease state extraction module 118 into a compact representation of the condition changes over time. FIG. 4 shows an exemplary disease state comparison 400 showing two enhanced images 410, 420 representing chest x-ray images taken at different time points, the comparison 400 also including temporal context information 430 regarding the temporal disease state since the previous image was taken. Although FIG. 4 shows the enhanced images 410, 420 side-by-side, in a second embodiment, the enhanced images 410, 420 may be displayed in nested fashion, i.e., one image behind another. The second embodiment may be used when more than two enhanced images are being compared so that the images are easily accessed but do not overwhelm the display.

The two images 410, 420 shown in the disease state comparison 400 have been generated by the classification and visualization module 116, and thus include the heat map layer, condition information, and exam date information, as described above. The first image 410 is the enhanced image 210 shown in FIG. 2, showing a right pleural effusion and an exam date along with a severity heat map. The second image 420, taken at a later date, has also been generated by the classification and visualization module 116. However, because the condition has been resolved, the heat map layer is completely shown as blue, i.e., no pixel in the x-ray image upon which the enhanced image 210 is based shows the right pleural effusion condition. Accordingly, there is no condition tag.

The temporal context information 430 shows that the condition has been completely resolved. This information 430 has been derived from the temporal disease extraction module 118, as shown with respect to FIG. 3.

Thus, the system 100 provides a compact representation of temporal trends in a condition with a visualization of the condition in images taken over a time span. A clinician such as an ICU physician may quickly identify the changes and ensure a coherence of interpretation between a radiologist and the physician. If the ICU physician's interpretation of the two images does not agree with the radiologist report, further tests may be quickly ordered to clarify the disagreement.

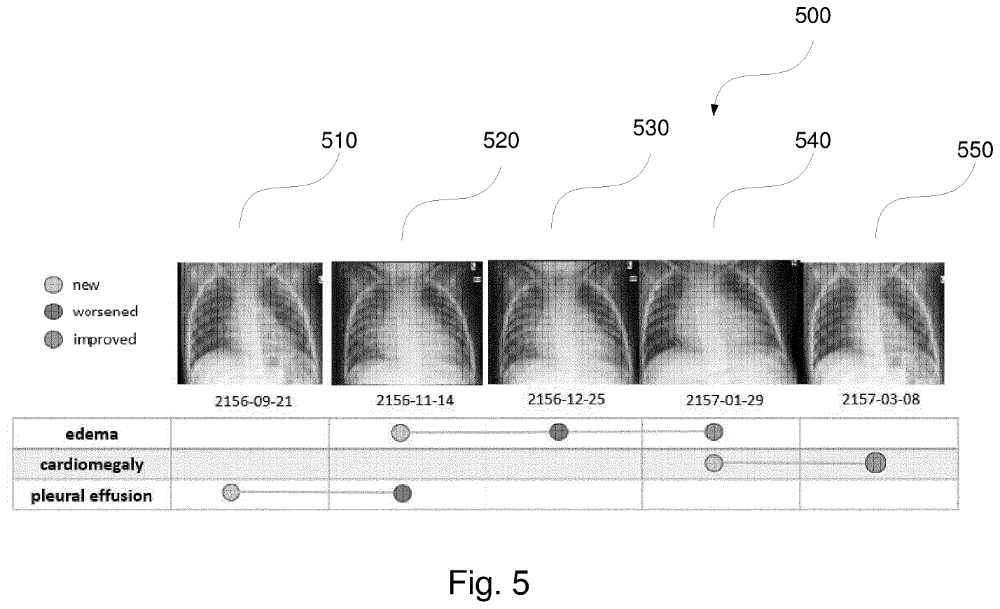

In another embodiment, the system 100 may analyze for any condition, rather than for a specific condition provided by e.g. the clinician. FIG. 5 shows an exemplary temporal progression 500 for a plurality of conditions, i.e., edema, cardiomegaly, and pleural effusion. A separate temporal trend is created for each condition shown in any one of the images 510-550. For example, the image 510 shows a pleural effusion condition as a “new” condition. The image 520 shows the pleural effusion condition “worsened,” as compared with the image 510, and an edema condition as “new.” The image 530 shows the edema condition “worsened,” as compared with the image 520. The image 540 shows the edema condition as “improved,” while a cardiomegaly condition is shown as “new,” as compared with the image 530. The image 550 shows the cardiomegaly condition as “improved,” as compared with the image 540. It is noted that the heat maps provided by the classification and visualization module 116 are omitted from the temporal progression 500, however, in another embodiment, they may be included. However, in a multi-condition temporal analysis including heat maps, a separate enhanced image is generated for each condition, showing the heat map representation of the specific condition.

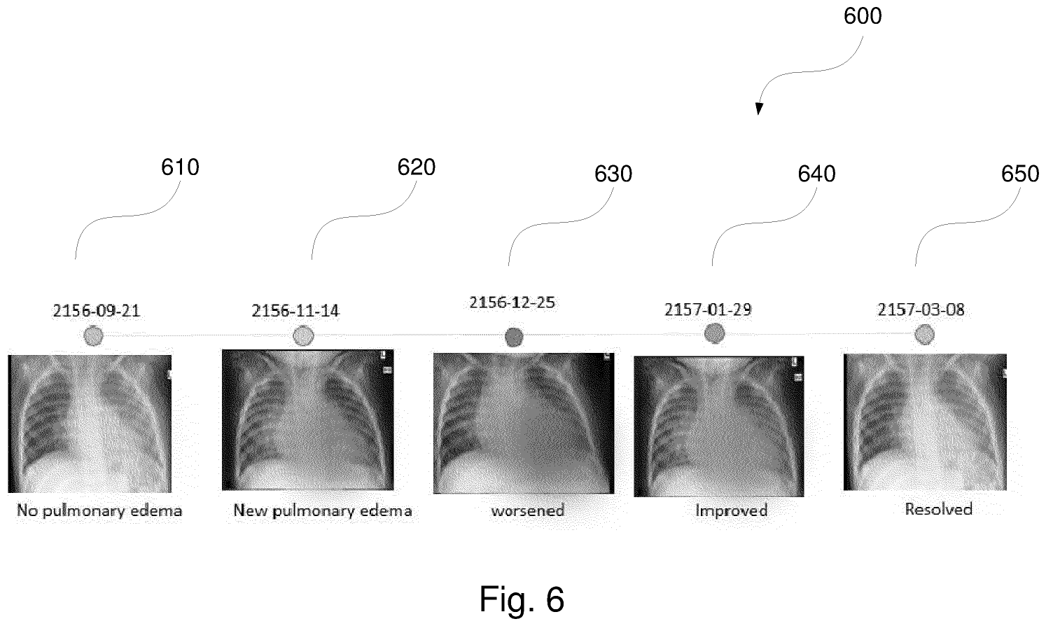

A third representation of temporal trends may also be generated by the system 100. FIG. 6 shows an exemplary temporal progression 600 of a condition from a time prior to the condition appearing to a time when the condition has been resolved.

The images 610-650 show x-ray images enhanced in an alternate manner than that described above. In the temporal progression 600, the images 610-650 have portions colored based on temporal state rather than based solely on the presence of the image, as will be described in more detail below. For example, the second image 620 shows a pulmonary edema that has not appeared in any prior image, while the third image 630 shows a worsened pulmonary edema (compared to the second image 620) and the fourth image 640 shows an improved pulmonary edema (compared to the third image 630). The first image 610 and the fifth image 650 show normal lungs, with no pulmonary edema.

The images 610-650 have all been analyzed by the classification and visualization module 116 to identify and score a potential presence of the disease in the pixels of the image, in a manner similar to that described above. However, instead of generating a heat map such as that shown in FIG. 2, the images 610-650 are distinguished with a color based on a change in condition between images. For example, the second image 620 has an area that is e.g. pink to show a new condition, while the third image 530 has an area that is e.g. red to show a worsened condition. The fourth image 640 has an area that is e.g. orange to show an improved condition, while the fifth image 650 has an area that is e.g. green to show a resolved condition, i.e. normal lungs. The first image 610, captured prior to the appearance of the pulmonary edema, may also have an area that is green to show an absence of the condition. Alternately, the first image 610 may not include any color.

Thus, the image analysis and scoring described for generating the heat map of FIG. 2 may also be used, however, the images shown in FIG. 6 are processed and enhanced differently to show a comparative color-coding. The images 610-650 may be labelled/annotated in a similar manner to that described with respect to FIG. 4.

The display of the various images described herein may be configured so that the images and the extracted information are positioned in any number of ways or permutations with respect to one another, e.g., varying horizontal, vertical, or mixed arrangements of windows, for example. Further, as mentioned previously, a nested display may be used instead of a side-by-side display.

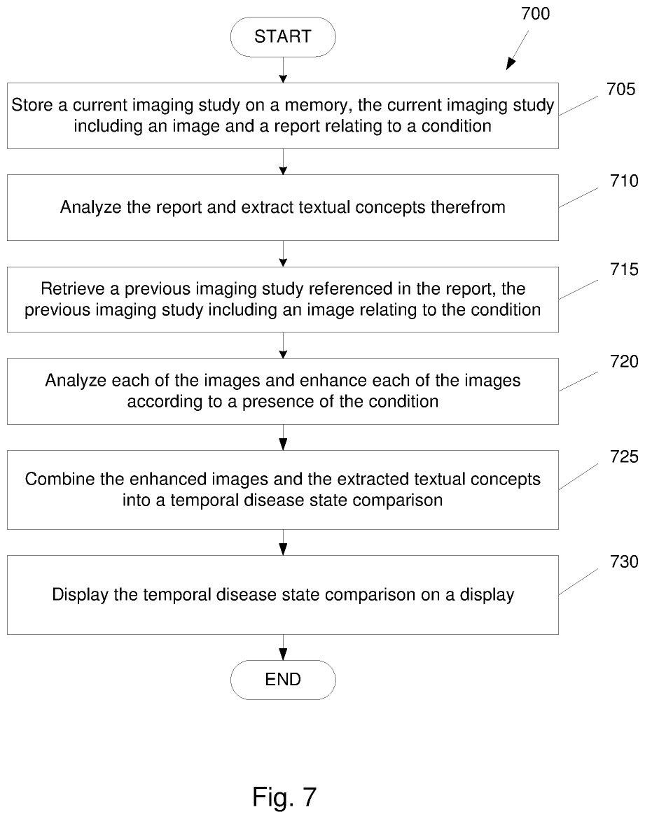

FIG. 7 shows a flow diagram 700 of a method for generating a temporal disease comparison according to various exemplary embodiments of the present disclosure.

In 705, a current imaging study, e.g. imaging study 110-1, is stored on the memory 104. As discussed previously, the memory 104 may store any number of imaging studies 110 for a patient, the imaging studies 110 relating to a single condition or any number of conditions. The current imaging study 110-1 includes at least one image 112-1 and an accompanying report 114-1, typically written by a radiologist.

In 710, the processor 102 analyzes the report 114-1 via the temporal disease state extraction module 118 and extracts textual concepts from the report 114-1 including a past exam date (to which the current exam is being compared), a current exam date, a condition being assessed and a temporal state of the condition (e.g. improved, worsened, etc.).

In 715, the processor 102 retrieves a previous imaging study, e.g. imaging study 110-2, from the memory 104. The previous imaging study 110-2 is the study referenced by the past exam date in the report 114-1. The previous imaging study 110-2 includes at least one image 112-2. The previous imaging study 110-2 may also include an accompanying report 114-2, however, this is not required for performing the presently described embodiment. In the presently described embodiment, the difference between only the two imaging studies 110-1 and 110-2 is being assessed, and this textual assessment may be found in the report 114-1 of the current imaging study 110-1. Even if the report 114-2 of the previous imaging study 110-2 contained temporal language relating to an imaging study even further back in time, e.g. a past imaging study 110-3, this temporal assessment comparing imaging studies 110-2 and 110-3 would be immaterial to the presently described embodiment.

In 720, the processor 102 analyzes the images 112-1 and 112-2 via the classification and visualization module 116. As previously described, the processor 102 may implement e.g. a CNN-based class activation mapping model to determine a presence of the condition in an image, e.g. images 112-1 and 112-2, and enhance each of the images 112-1 and 112-2 with a heat map indicating a probability of the presence of the condition in the images 112-1 and 112-2, on a pixel-by-pixel basis. Additionally, the classification and visualization module 116 tags the enhanced images with a condition and a date of the exam, as previously discussed.

In 725, the processor 102 combines the outputs of the modules 116 and 118 into a temporal disease state comparison, e.g. disease state comparison 400 shown in FIG. 4. The disease state comparison 400 includes the enhanced versions of the images 112-1 and 112-2, e.g. the images 410 and 420 shown in FIG. 4. The images 410 and 420 include a heat map indicating the probability of the presence of the analyzed condition, the date tags and the condition tags (if the condition is present). In addition, the temporal disease state comparison includes the temporal language extracted from the report 114-1, the temporal language relating to the evolution of the condition between the current imaging study 110-1 and the previous imaging study 110-2.

In 730, the processor 102 displays the temporal disease state comparison on the display 108 for quick, compact viewing by a clinician e.g. an ICU clinician.

The exemplary embodiments described above, including the image analysis and the associated textual labelling, may be used as training data for further modeling. For example, neural networks generally require an analysis and a label to train the network. Thus, the temporal state disease comparisons may be used to train e.g. disease progression models or temporal state change prediction models. For example, a temporal state change prediction model may be used to predict an evolution of a disease that has not yet been resolved, based on e.g. training data including the evolution of a disease that has been fully resolved.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A method, comprising:
analyzing a first image to determine whether an abnormal medical condition is present in the first image;
enhancing the first image with a first heat map indicating a probability of a presence of the abnormal medical condition in an area of the first image;
analyzing a second image to determine whether the abnormal medical condition is present in the second image;
enhancing the second image with a second heat map indicating a probability of a presence of the abnormal medical condition in an area of the second image;
analyzing a text-based report corresponding to the second image to extract information corresponding to the second image and changes in the abnormal medical condition between the first image and the second image; and
displaying the enhanced first image, the enhanced second image and the extracted information on a single display.

2. The method of claim 1, wherein each of the first and second images is analyzed on a pixel-by-pixel basis to determine whether the abnormal medical condition is present in the image, wherein each pixel is assigned a score based on a convolutional neural network (CNN) based class activation mapping model.

3. The method of claim 2, wherein each of the first and second heat map is generated based on the score of each of the pixels.

4. The method of claim 1, further comprising:
tagging at least one of the first and second images, wherein the tagging includes a textual indication of the abnormal medical condition.

5. The method of claim 1, further comprising:
receiving, via a user interface, an indication of the abnormal medical condition for which the first and second images are to be analyzed.

6. The method of claim 1, wherein the text-based report is analyzed using natural language processing (NLP).

7. The method of claim 1, wherein the extracted information comprises an exam date of the first image, an exam date of the second image, and an identification of the abnormal medical condition.

8. The method of claim 1, wherein the changes in the abnormal medical condition between the first image and the second image indicates one of a plurality of temporal states of the abnormal medical condition, the plurality of temporal states comprising worsened, improved, resolved and unchanged.

9. The method of claim 1, further comprising:
analyzing a third image to determine whether the abnormal medical condition is present in the third image;
enhancing the third image with a third heat map indicating a probability of a presence of the abnormal medical condition in an area of the third image; and
displaying the enhanced first image, the enhanced second image, the enhanced third image and the extracted information on a single display, wherein the enhanced first image, the enhanced second image and the enhanced third image are displayed in date order on the single display.

10. A computer readable storage medium comprising a computer program that when executed by a processor, performs the method of claim 1.

11. A system, comprising:
a memory configured to store a plurality of imaging studies, at least a portion of the imaging studies comprising an image and a corresponding text-based image report;
a processor configured to perform operations comprising,
analyzing a first image of a first one of the plurality of imaging studies to determine whether an abnormal medical condition is present in the first image,
enhancing the first image with a first heat map indicating a probability of a presence of the abnormal medical condition in an area of the first image,
analyzing a second image of a second one of the plurality of imaging studies to determine whether the abnormal medical condition is present in the second image,
enhancing the second image with a second heat map indicating a probability of a presence of the abnormal medical condition in an area of the second image,
analyzing a corresponding text-based report of the second one of the imaging studies to extract information corresponding to the second image and changes in the abnormal medical condition between the first image and the second image; and
a display configured to display the enhanced first image, the enhanced second image and the extracted information.

12. The system of claim 11, wherein each of the first and second images is analyzed on a pixel-by-pixel basis to determine whether the abnormal medical condition is present in the image, wherein each pixel is assigned a score based on a convolutional neural network (CNN) based class activation mapping model.

13. The system of claim 12, wherein each of the first and second heat map is generated based on the score of each of the pixels.

14. The system of claim 11, wherein the processor is further configured to perform operations comprising:
tagging at least one of the first and second images, wherein the tagging includes a textual indication of the abnormal medical condition.

15. The system of claim 11, further comprising:
a user interface configured to receive an indication of the abnormal medical condition for which the first and second images are to be analyzed.

16. The system of claim 11, wherein the text-based report is analyzed using natural language processing (NLP).

17. The system of claim 11, wherein the extracted information comprises an exam date of the first image, an exam date of the second image, and an identification of the abnormal medical condition.

18. The system of claim 11, wherein the changes in the abnormal medical condition between the first image and the second image indicates one of a plurality of temporal states of the abnormal medical condition, the plurality of temporal states comprising worsened, improved, resolved and unchanged.

19. The system of claim 11, wherein the processor is further configured to perform operations comprising:
analyzing a third image to determine whether the abnormal medical condition is present in the third image; and
enhancing the third image with a third heat map indicating a probability of a presence of the abnormal medical condition in an area of the third image;
wherein the display is further configured to:
display the enhanced first image, the enhanced second image, the enhanced third image and the extracted information on a single display, wherein the enhanced first image, the enhanced second image and the enhanced third image are displayed in date order on the single display.
